(19) United States
(12) Patent Application Publication
(10) Pub. No.: US 2006/0223719 A1
(43) Pub. Date: Oct. 5, 2006

(54) Method of improving properties of hydroforming fluids using overbased sulfonate

(76) Inventors: Igor Riff, Skillman, NJ (US); Michael T. Costello, Southington, CT (US)

Correspondence Address: Daniel Reitenbach;CHEMTURA CORPORATION, Benson Road, Middlebury, CT, 06749, US

(21) Appl. No.: 11/398,418

(22) Filed: Apr. 4, 2006

Related U.S. Application Data
(60) Provisional application No. 60/668,066, filed on Apr. 5, 2005.

(51) Int. Cl. C07C 309/62

(52) U.S. Cl. 508/390; 508/460; 508/545

(57) ABSTRACT

A method for improving lubricants employed in hydroforming processes is disclosed wherein said method comprises adding to said lubricants at least one overbased detergent and at least one friction modifier.

This application claims priority to U.S. Provisional Application No. 60/668,066, which was filed on Apr. 5, 2005.

### BACKGROUND OF THE INVENTION

1. Field of the Invention

The present invention relates to lubricants used in metal forming processes and, in particular, to lubricants used in hydroforming processes.

2. Description of Related Art

Metal parts that are machined require lubrication to reduce equipment wear. These include such operations as bending, swaging, tapping, drawing, and hydroforming. Hydroforming is a particularly important process when a relatively complex part is manufactured. In tube hydroforming, a workpiece is placed in a tool cavity, where the geometry of the cavity corresponds to the external geometry of the part. The tool cavity is closed by a ram movement of the press, while an aqueous fluid is simultaneously pumped into the ends of the tube along the axis. As the internal pressure of the tube increases, the tube expands to fill the internal cavity to form the desired part. The advantages of this process over conventional drawing operations is the ability to form deeper parts, which can lead to fewer total parts and welds that results in a lighter overall part weight.

There are three types of lubricants involved in tube hydroforming (THF). These include the bending lube, pressure side aqueous fluid, and the die-side lubricant. The bending lubricant is used on the inside of the tube to prebend the part prior to the THF operation. The pressure side aqueous fluid is used to transmit pressure to the inside of the tube and, while lubricity is not critical for this fluid, the corrosion protection and high-pressure stability play a vital role. Finally, the die-side lubricant is the primary forming fluid in the THF operation and provides lubricity between the workpiece and the die. The demands of the die side lubricant vary widely depending on the internal pressure of the aqueous fluid. Additionally, as the complexity of the part increases, so does the demand on the die-side lubricant.

Lubrication and friction control in the hydroforming process are essential to allow the tube material to slide within the die as the internal pressure rises. Without adequate lubrication, hydroformed parts can neck or fracture prematurely during the forming operation. Proper selection of the lubricant depends on many factors, including: the material used for forming, sump maintenance, cleanability, rust inhibition, and environmental acceptance. For the hydroforming process, the coefficient of friction is a function of pressure, speed, sliding distance, material properties, and the surface finishes of both the hydroforming die and the tube. It has been found there are distinct regions of lubrication for the hydroforming process, which include a guiding zone and an expansion zone. In these two regions, hydroforming tests display an antagonism, where, as the expansion zone performance increases, the guiding zone performance decreases.

U.S. Patent Application No. 2003/0181340 A1 discloses a hydroforming process for metal parts that uses liquid-film and solid-film lubricants. The lubricants used in the invention are particularly useful for die-side lubrication. The process includes a step in which a ductile metal part is over-coated with either the liquid-film or solid-film lubricant. The liquid lubricants preferably include an oil and, optionally, a surfactant. The solid lubricants preferably include a hard wax and, optionally, a surfactant.

Ahmetoglu, M. et al., SAE International Congress and Exposition, Detroit, Mich., 1999, 199-01-0675 reviewed the fundamentals of tube hydroforming technology and discussed how various parameters, such as tube material properties, pre-form geometry, lubrication, and process control affect product design and quality. In addition, the relations between process variables and achievable part geometry were discussed. Finally, using examples, the status of the current technology and critical issues for future development were reviewed.

Dalton, G., Automotive Tube Conference, Apr. 26-27, 1999 discussed the role of lubricants in hydroforming. Lubricants are used in bending, between the tube and hydroforming die, and in the pressure media. It was stated that these lubricants must be compatible with each other, control friction and die wear, and permit welding and painting, and that proper selection of lubricants will ensure the hydroforming process is capable and cost effective. The important variables, how to evaluate them, and factors to consider in choosing the best lubricants were outlined.

Bartley, G. et al., Light Metal Age, 58(7,8):24, 26, 28, 30, 32, 34, 36-37 (2000) described hydroforming process basics, equipment, parameters, and benefits to the automotive industry, with special emphasis on the use of aluminum extrusions as workpiece materials. Then current hydroformed aluminum projects were also presented.

Koc, M. et al., Journal of Materials Processing Technology, 108:384-393 (2001) summarized a technological review of the hydroforming process from its early years to very recent dates on various topics, such as material, tribology, equipment, tooling, and the like.

Khodayari, G. et al, Analyzing Tubes, Lubes, Dies and Friction TPJ—The Tube and Pipe Journal, Oct. 10, 2002, compared and correlated two tests, a common bench test (twist compression) and a straight-tube corner-fill test that simulate hydroforming to find the coefficient of friction.

Ngaile, G. et al., Journal of Materials Processing Technology, 146(1): 108-11 (2004) discussed the lubrication mechanisms that occur at the tool-workpiece interface for the transition and expansion zones. Suitable lubrication systems for the transition and expansion zones were reviewed based on the mechanics of deformation and material flow at the interface. Details of two model tests for evaluating the performance of tube hydroforming lubricants and die coatings were given. The optimization of die geometries for the model tests was based on sensitivity analysis through the finite element method together with experimental verification. The details of these tests were given and their development was also discussed.

Ngaile, G. et al., Journal of Materials Processing Technology, 146(1):116-123 (2004) presented two model tests for evaluating lubricant performance under realistic tribological conditions occurring in the transition and expansion zones of a tube hydroforming process. The model test for the transition zone was based on the limiting dome height (LDH) test principle. For the expansion zone, a pear-shaped tube expansion test (PET) developed by the authors was employed. Four lubricants were tested and ranked based on (a) dome wall thinning behavior (for LDH), (b) tube wall thinning, tube protrusion height, tube bursting pressure (for PET), and (c) surface topography. Friction coefficients for the lubricants were estimated by matching experimental and FE results.

Tung, S. C. et al., Tribology International, 37(7):517-536 (2004) provided an overview of various lubrication aspects of a typical powertrain system including the engine, transmission, driveline, and other components, as well as the integration of these lubrication and surface engineering concepts into a unified automotive powertrain system. It was pointed out that recent industrial developments include high strength and high density composite materials, high volume liquid molding and hydroforming technology, structural adhesive bonding, and the ability to mold large structural components.

Overbased metal sulfonates are commonly used in lubricating oil compositions as rust inhibitors and detergents. It is highly desirable for such sulfonates to provide neutralization capacity for acids formed in engine combustion without too rapid loss in alkalinity.

The use of normal salts of petroleum sulfonic acids as additives for lubricating oil compositions is well known. During World War II, normal metal sulfonates that were derived from mahogany or petroleum sulfonic acids were employed as detergent additives in internal combustion engine crankcase oils. Calcium or barium was employed as the metal in such sulfonates. Subsequently, sulfonate products which contained up to twice as much metal as the corresponding metal sulfonate were found to have improved detergent power and ability to neutralize acidic contaminants and, hence, were used in the place of the normal sulfonates. More recently, fully oil-soluble sulfonates containing from three up to twenty or more times as much metal as a corresponding normal metal sulfonates have been developed. These high based sulfonates have been identified as “overbased”, “superbased”, and “hyperbased”.

Over the years, numerous methods for preparing overbased sulfonates have been disclosed. In general, such overbased sulfonates have been prepared by mixing a promoter and a solvent with a normal sulfonate and an excessive amount of a metallic base of either an alkali metal or an alkaline earth metal, heating the resulting mixture, carbonating the resulting reaction mass with sufficient carbon dioxide to increase the amount of metal base colloidally dispersed as metal carbonate in the resulting product, and then filtering the resulting material. Several of the particular methods are summarized in the following paragraphs.

U.S. Pat. No. 3,488,284 discloses a process for preparing oil soluble basic metal complexes formed by treating an oil soluble sulfonic acid with a metal base in the presence of an acidic gas and an alcohol promoter. The process is said to produce oil soluble metal containing compositions having “metal ratios” i.e., ratios of total metal in the product to the amount of metal which is in the form of the normal salt of the sulfonic acid, of up to about 7 or more.

U.S. Pat. No. 3,446,736 discloses the formation of a calcium sulfonate-calcium carbonate product by preparing a calcium carbonate reagent in methanol and reacting such reagent with a sulfonic acid or sulfonate salt. For example, the calcium carbonate reagent prepared by carbonating a suitable calcium inorganic compound in methanol with carbon dioxide at a temperature below about 30° C. was intermixed with a solution of a sulfonic acid or sulfonate in mineral oil. Then the resulting mixture was heated to a temperature above the boiling point of methanol to facilitate reaction and to remove methanol by distillation.

U.S. Pat. No. 3,496,105 discloses in preparing an overbased material, that the compound to be overbased, e.g., an oil-soluble sulfonic acid or a sulfonate, a substantially inert organic solvent, a Group II metal base, an alcoholic or phenolic promoter, and an acidic material, such as CO₂, H₂S, SO₂, or SO₃, are mixed together. The temperature at which the acidic material is contacted with the remainder of the reaction mass depends upon the promoting agent employed.

U.S. Pat. No. 3,907,691 discloses that the overbasing process can be conducted conveniently by mixing a neutral metal sulfonate and an inert hydrocarbon solvent, adding an alkaline earth metal base and an alkanol having one to four carbon atoms to the resulting mixture at a temperature and pressure effective in retaining most of the alkanol charged, contacting the reaction mixture with carbon dioxide until its absorption into the mixture ceases or substantially decreases, and heating the resulting product to strip out the residual alkanol and water of reaction.

U.S. Pat. No. 4,137,184, discloses the preparation of a Group II metal sulfonate carbonated with carbon dioxide at ambient temperature in the presence of solvent, methanol, and a Group II metal hydroxide for a period of time. The carbonated material is then heated to a higher temperature to remove solvent, methanol, and water. The carbon dioxide is passed through the mixture at a rate such that all the carbon dioxide is taken up without off-gas.

U.S. Pat. No. 4,328,111 discloses compositions of matter that comprise the reaction product of a basic compound comprising an overbased metal sulfonate, phenate, or mixtures thereof, with an acidic compound comprising organic carboxylic acid, organic carboxylic acid anhydride, phosphoric acid, phosphoric acid ester, thiophosphoric acid ester, or mixtures thereof.

U.S. Pat. No. 4,880,550 discloses a method for preparing a carbonate overbased calcium sulfonate, which method comprises the steps of: (1) forming an initial mixture of a lower molecular weight alkanol, an alkyl or alkaryl substituted sulfonic acid or sulfonate compound, a diluent and a solvent; (2) adding a basic calcium compound to the initial mixture to form a second mixture in which the amount of added calcium is at least about ten times the amount necessary to form a neutral calcium sulfonate; (3) heating the second mixture to reflux temperature; (4) carbonating the second mixture at said reflux temperature to form a carbonated product while simultaneously and continuously removing water produced by the carbonation reaction; (5) after carbonation is stopped, heating the carbonated product to an elevated temperature sufficient to remove the alkanol; and (6) removing solids and solvent from the carbonated product.

GB 2,082,619A discloses a process for the preparation of a highly basic calcium sulfonate, wherein a mixture of an oil-soluble sulfonic acid or alkaline earth metal sulfonate, calcium hydroxide, an alcohol having from one to four carbon atoms, an aromatic or aliphatic hydrocarbon solvent, and water is formed; the mixture is carbonated with carbon dioxide at a temperature that is maintained within the range of 25° C. to 30° C. until just prior to complete reaction of carbon dioxide with the calcium hydroxide, at which time further calcium hydroxide is added; carbonation is completed at a temperature within the range of from about 50° C. to 100° C. where from 5 wt % to 20 wt % of water, based on the weight of calcium hydroxide, is used; and the resulting mixture is heated to an elevated temperature, e.g., above 130° C. to remove water, alcohol, and solvent.

The disclosures of the foregoing are incorporated herein by reference in their entirety.

### SUMMARY OF THE INVENTION

The present invention relates to lubricants used in hydroforming processes and, especially, to improving such lubricants by means of additives, in particular, a combination of overbased detergents, e.g., over based sulfonates, and friction modifiers.

In one aspect, the present invention is directed to a method for improving lubricants employed in hydroforming processes comprising adding to said lubricants at least one overbased detergent and at least one friction modifier. The overbased detergent is preferably an overbased sulfonate, carboxylate, phenate, salicylate, or a mixture thereof, more preferably and overbased sulfonate, and the friction modifier is preferably a fatty acid ester, a reaction products of a fatty acid ester and an ethoxylated amine, an overbased carboxylic acid, a molybdenum dithiocarbamate derivative, or a mixture of the foregoing.

In another aspect, the present invention is directed a lubricant for use in hydroforming processes wherein said lubricant comprises at least one overbased detergent and at least one friction modifier, as described above.

In still another aspect, the present invention is directed to an improvement in a method for hydroforming metal tubes, wherein the improvement comprises employing in said method a lubricant comprising at least one overbased detergent and at least one friction modifier, as described above.

### DESCRIPTION OF THE PREFERRED EMBODIMENTS

The overbased detergent additives of the present invention are preferably alkaline earth metal sulfonates, more preferably an overbased calcium sulfonate, an overbased magnesium sulfonate, an overbased barium sulfonate, or a mixture or two or more of the foregoing.

Overbased alkaline earth metal sulfonates may be obtained by overbasing a neutral alkaline earth metal sulfonate to produce an alkaline earth metal carbonate, such as calcium carbonate and magnesium carbonate, or an alkaline earth metal borate, such as magnesium borate.

The base number of the metal sulfonate is not particularly limited; however, the base number is normally in the range of from about 5 to about 500 mg KOH/g, preferably from about 300 to about 400 mg KOH/g.

In general, the process of preparing the overbased calcium sulfonates of the present invention comprises reacting a solution of calcium sulfonate or sulfonic acid (for convenience, the following discussion will focus on calcium compounds, but those skilled in the art will readily comprehend that, by analogy, the process can be applied to other alkaline earth compounds, as well as to mixtures thereof) in oil with a slurry of calcium oxide or hydroxide and bubbling carbon dioxide through the reaction mixture, thereby incorporating an excess of calcium carbonate into the calcium sulfonate, which confers the desired reserve alkalinity to the product. In this process, it has been found advantageous to add a low molecular weight alcohol, such as methanol, and water to promote the formation of a micellar dispersion of calcium carbonate.

Calcium hydroxide when used commercially as the sole reserve alkalinity agent in the reaction mixture is used in substantial excess in order to achieve a high TBN product.

A dispersant is an optional component of the process and product for the calcite overbased additive. A preferred dispersant is the reaction product of hydrocarbyl-substituted succinic acid or anhydride with amines containing at least one primary or secondary amino nitrogen, e.g., the polyalkylene polyamines fulfill this requirement as do the substituted polyalkylene polyamines, and for that matter, ammonia. The bis-succinimides are also useful as optional dispersants. The bis-succinimides are prepared by the reaction of hydrocarbyl-substituted succinic acid or anhydride with an amine containing at least two primary and/or secondary nitrogens. Such bis-succinimides are, for example, the polyisobutenyl bis-succinimides of ethylene diamine, diethylene traimine, or triethylene tetramine, or tetraethylene pentamine or N-methyldipropylene triamine, etc. (See, for example, U.S. Pat. No. 3,438,899). The various above-described dispersing agents can be used alone or in mixtures.

Examples of friction modifiers that can be used in combination with the overbased sulfonate additives of the present invention include fatty acid esters and amides, organo molybdenum compounds, molybdenum dialkylthiocarbamates, molybdenum dialkyl dithiophosphates, and the like. Glycerol monooleate and oleic acid reacted with triethanol amine are particularly preferred.

The lubricant additives of this invention can be used in combination with other additives typically found in hydroforming fluids, and such combinations may, in fact, provide synergistic effects toward improving desired properties. Such additives include, but are not limited to, dispersants, rust inhibitors, anti-oxidants, biocides, extreme pressure (EP), antiwear (AW), and the like.

Examples of dispersants include polyisobutylene succinimides, polyisobutylene succinate esters, Mannich Base ashless dispersants, and the like.

An example of a rust inhibitor is polyoxyalkylene polyols, and the like.

Examples of antioxidants include alkylated diphenylamines and N-alkylated phenylenediamines. Secondary diarylamines are well known antioxidants and there is no particular restriction on the type of secondary diarylamine that can be used. Further examples of the antioxidant types include the hindered phenolic type, oil soluble copper compounds, and the like.

Examples of biocides include, but are not limited to, triazines, phenols, morpholines, “formaldehyde releasers (compounds that will hydrolyze into formaldehyde and other non-persistent fragments in aqueous solution),” azoniatricylodecanes, omadines, oxazolidines, and the like.

The present invention is directed to the use of overbased sulfonate additives and organic friction modifiers that improve either the expansion zone or the guiding zone of hydroforming applications or both.

In the discussion that follows, the following designations are employed:

| Name | Description |
|---|---|
| Reference #1 | commercial hydrofroming lubricant (liquid, 1200 cSt) |
| Reference #2 | commercial hydrofroming lubricant (solid) |
| C300C | overbased calcium sulfonate (crystalline 300 TBN) |
| C400A | overbased calcium sulfonate (amorphous 400 TBN) |
| M400A | overbased magnesium sulfonate (400 TBN) |
| B70A | overbased barium sulfonate (70 TBN) |
| GMO | glycerol monooleate |
| OA/TEA | oleic acid reacted with triethanol amine |
| COB40 | overbased calcium carboxylate (from tall oil) |
| Mo(DTC) | mixed thio-acid amide molybdenum complex |

Guiding (or Feeding) Zone—The area where the part is fed into the die toward the zone where the part is expanded. In this area is mostly sliding and requires a low coefficient of friction.

Expansion Zone—The area where the part is expanded to an irregular shape and the surface area increases while the part thickness decreases.

Transition Zone—The area between the guiding zone and expansion zone. This area requires a low coefficient of friction and is a combination of sliding and expansion.

### Test Methods

Twist-Compression (Transition Zone) Test—The twist-compression rig uses a D2 steel annular specimen rotating against a lubricant coated (9,978±78 mg/ft² coating weight flooded conditions) flat cold rolled steel (CRS) specimen. The test was run at an interface pressure of 5000±250 psi and a rotational speed of 8.9 rpm. The samples were tested for a maximum of 1000 seconds or until lubricant failure. Lubricant failure is defined as the time necessary for the coefficient of friction (COF) to reach 0.20 or 0.30. Four replicates were run on each test. In hydroforming applications, the longer the time to failure the better the lubricant performs in the transition zone (the area between the expansion and guiding zone).

Guiding Zone Test—The guiding zone test uses a tubular specimen of 101 mm (4.0 inches) length, 57 mm (2.25 inches) diameter, and 2 mm (0.0787 inch) wall thickness cut from hot rolled low carbon steel 1010 material inserted into a 160-ton hydraulic press at a ram speed of 65 mm/sec with an internal pressure of 600 bar. The coefficient of friction is then measured for a sliding distance of 140 mm. The average surface roughness of the tubular specimen before the test was Rₘₐₓ=9.4 μm and Rₐ=1.1 μm. The test lubricants were applied to the specimen just prior to the testing (3 to 5 elapsed minutes between application and testing) with a small paintbrush. Although the test rig was not in a controlled environment, the temperature and humidity were approximately 75° F. and 15%, respectively. In hydroforming applications, the lower the coefficient of friction the better the lubricant performs in the guiding (or feeding) zone.

Expansion Zone Test—The expansion zone test uses a tubular specimen of 250 mm (10 inches) length, 57 mm (2.25 inches) diameter, and 2 mm (0.0787 inch) wall thickness cut from hot rolled low carbon steel 1010 material inserted into a pear-shaped dye and pressurized until the tube burst. The die inserts are designed for testing a tube 57 mm in diameter and 100 mm in effective length. Owing to the lower expansion of the steel used in this test, the burst pressure was measured instead of the bulge height. The average surface roughness of the tubular specimen before the test was Rₘₐₓ=9.4 μm and Rₐ=1.1 μm. The test lubricants were applied to the specimen just prior to the testing (3 to 5 elapsed minutes between application and testing) with a small paintbrush. Although the test rig was not in a controlled environment, the temperature and humidity were approximately 75° F. and 15%, respectively. In hydroforming applications, a higher burst pressure is better than a lower one and a burst closer to the center of the tubular specimen is more desired.

U.S. Patent Application No. 2003/0181340 A1 discloses additional details of the test methods used in this invention.

### EXAMPLES

A preliminary comparison of the performance of a hydroforming formulation using overbased calcium sulfonate detergents and organic friction modifiers is presented in Table 1 (Examples 1-6).

TABLE 1

| | Example | | | | | |
|---|---|---|---|---|---|---|
| | 1 | 2 | 3 | 4 | 5 | 6 |
| Reference Lube #1 | 100 | | | | | |
| Reference Lube #2 | | 100 | | | | |
| C300C | | | 10 | | 10 | |
| C400A | | | | 10 | | 10 |
| GMO | | | | | 1 | 1 |
| 750SUS oil | | | 90 | 90 | 89 | 89 |

Guiding Zone

| | | | | | | |
|---|---|---|---|---|---|---|
| Coefficient of Friction | 0.0140 | — | 0.0355 | 0.0360 | 0.0370 | 0.0375 |
| (20 mm Sliding Length) | | | | | | |
| Coefficient of Friction | 0.0150 | — | 0.0370 | 0.0430 | 0.0400 | 0.0350 |
| (50 mm Sliding Length) | | | | | | |
| Coefficient of Friction | 0.0200 | — | 0.0460 | 0.0550 | 0.0475 | 0.0410 |
| (80 mm Sliding Length) | | | | | | |
| Average Surface | 0.6 | — | 0.7 | 0.7 | 0.7 | 0.7 |
| Roughness (um) | | | | | | |

Expansion Zone

| | | | | | | |
|---|---|---|---|---|---|---|
| Pressure (Bar) | 502 | 548 | 500 | 524 | 530 | 497 |
| Average Surface | — | — | — | — | — | — |
| Roughness (um) | | | | | | |

Examples 1-2 demonstrate the baseline performance of both liquid Reference Lube #1 and solid Reference Lube #2 commercial products for hydroforming. The solid product, Reference Lube #2, performs better than the liquid product, Reference Lube #1 in the expansion zone test.

Examples 3-4 demonstrate the performance in the expansion and guiding zone when overbased calcium sulfonate detergent is added to the base oil. The amorphous overbased calcium sulfonate, C400A, is a credit in the expansion zone test, while the crystalline overbased calcium sulfonate, C300C, is a credit in the guiding zone test.

Examples 5-6 demonstrate the effect of the addition of an organic friction modifier (such as glycerol monooleate or GMO) to the performance in the guiding zone and expansion zone test. It demonstrates an antagonistic effect where the organic friction modifier is credit in the performance of crystalline product in the expansion zone, while it is a debit in the performance in the guiding zone. For the amorphous material the effect is opposite, where the expansion zone performance is degraded and the guiding zone performance is improved.

Since the effects of the additives are antagonistic in these tests, it is difficult to improve both simultaneously. In order to improve the performance of a hydroforming lubricant, a balance must be struck between the additives where both the expansion and guiding zone tests are optimized.

In Table 2, the next set of examples (7-16) are blends that were made to determine the antagonisms of the additives with a formulated hydroforming fluid using the expansion zone, guiding zone, and twist compression (transition zone) tests.

TABLE 2

| | Example | | | | | | | | | |
|---|---|---|---|---|---|---|---|---|---|---|
| | 7 | 8 | 9 | 10 | 11 | 12 | 13 | 14 | 15 | 16 |
| Reference Lube #1 | 99 | 99 | 99 | 99 | 100 | 92.5 | 90 | 90 | 90 | 91.5 |
| C300C | | | | | | | 10 | | | |
| C400A | | | | | | 7.5 | | | | 7.5 |
| M400A | | | | | | | | 10 | | |
| B70A | | | | | | | | | 10 | |
| GMO | 1 | | | | | | | | | 1 |
| OA/TEA | | 1 | | | | | | | | |
| COB40 | | | 1 | | | | | | | |
| Mo(DTC) | | | | 1 | | | | | | |

Guiding Zone

| | | | | | | | | | | |
|---|---|---|---|---|---|---|---|---|---|---|
| Coefficient of Friction | 0.0212 | 0.0265 | 0.0200 | 0.0231 | 0.0207 | 0.0215 | 0.0204 | 0.0209 | 0.0216 | 0.0219 |
| (20 mm Sliding Length) | | | | | | | | | | |
| Coefficient of Friction | 0.0189 | 0.0253 | 0.0178 | 0.0196 | 0.0189 | 0.0189 | 0.0170 | 0.0192 | 0.0208 | 0.0214 |
| (50 mm Sliding Length) | | | | | | | | | | |
| Coefficient of Friction | 0.0230 | 0.0265 | 0.0192 | 0.0223 | 0.0203 | 0.0207 | 0.0190 | 0.0212 | 0.0244 | 0.0256 |
| (80 mm Sliding Length) | | | | | | | | | | |
| Average Surface | 0.8 | 0.9 | 0.7 | 0.8 | 0.8 | 0.7 | 0.9 | 0.9 | 0.7 | 0.8 |
| Roughness (um) | | | | | | | | | | |

Expansion Zone

| | | | | | | | | | | |
|---|---|---|---|---|---|---|---|---|---|---|
| Pressure (Bar) | 505 | 507 | 500 | 504 | 499 | 506 | 498 | 512 | 492 | 501 |
| Average Surface | 0.7 | 0.9 | 0.9 | 0.8 | 1.0 | 0.9 | 1.1 | 0.8 | 0.9 | 0.9 |
| Roughness (um) | | | | | | | | | | |

Twist Compression (Transition Zone)

| | | | | | | | | | | |
|---|---|---|---|---|---|---|---|---|---|---|
| COF = 0.2 (sec) | 360 | 312 | 279 | 279 | 244 | 246 | 260 | 437 | 341 | 179 |
| COF = 0.3 (sec) | 398 | 374 | 326 | 302 | 300 | 261 | 291 | 490 | 387 | 192 |

Examples 7-11 demonstrate the effect of various organic friction modifiers on the hydroforming fluid.

Example 7 demonstrates the effect of adding 1 wt. % glycerol monooleate, GMO, to the Reference Lube #1. The performance in the expansion zone test and twist compression (transition zone) test is improved.

Example 8 demonstrates the effect of adding 1 wt. % of the product of the reaction of one mole of oleic acid reacted with triethanol amine, OA/TEA, to the Reference Lube #1. The performance in the expansion zone test and twist compression test (transition zone) is improved.

Example 9 demonstrates the effect of adding 1 wt. % of overbased calcium carboxylate, COB40, to the Reference Lube #1. The performance in the guiding zone and twist compression test (transition zone) test is improved, while there is no harm in the expansion zone test.

Example 10 demonstrates the effect of adding 1 wt. % of the mixed thio-acid amide molybdenum complex, Mo(DTC), to the Reference Lube #1. The performance in the expansion zone test and twist compression (transition zone) test is improved.

Example 11 demonstrates the baseline performance of commercial hydroforming fluid, Reference Lube #1, in the guiding zone, expansion zone, and twist compression (transition zone) testing.

The addition of an organic friction modifier improves both the expansion zone test and twist compression (transition zone) test, while the addition of the overbased calcium carboxylate which is a combination of organic friction modifier and overbased detergent improves the guiding zone results as well.

Examples 12-15 demonstrate the effect of various overbased sulfonate detergents on guiding zone, expansion zone, and twist compression (transition zone) testing.

Example 12 demonstrates the effect of adding 7.5 wt. % of crystalline overbased calcium sulfonate, C300C, to the Reference Lube #1. The performance in the expansion zone and twist compression (transition zone) tests is improved without a large debit in the guiding zone test.

Example 13 demonstrates the effect of adding 10 wt. % of amorphous overbased calcium sulfonate, C400A, to the Reference Lube #1. The performance in the guiding zone test is improved without a large debit in the expansion zone and twist compression (transition zone) tests.

Example 14 demonstrates the effect of adding 10 wt. % of amorphous overbased magnesium sulfonate, M400A, to the Reference Lube #1. The performance in the expansion zone and twist compression (transition zone) tests is improved without a large debit in the guiding zone test.

Example 15 demonstrates the effect of adding 10 wt. % of amorphous overbased barium sulfonate, B70A, to the Reference Lube #1. The performance in the twist compression (transition zone) tests is improved, but there is a large debit in the guiding zone test.

Example 16 demonstrates the effect of adding 7.5 wt. % of amorphous overbased calcium sulfonate, C400A, and 1.0 wt. % of the organic friction modifier glycerol monooleate, GMO, to Reference Lube #1. There is an antagonism between these two additives which demonstrates a debit in guiding zone and twist compression (transition zone) testing and no effect in the expansion zone test.

It was found that overbased sulfonate detergents could improve the performance in the expansion zone test, and twist compression test (transition zone), and guiding zone test, depending on the composition of the formulation In view of the many changes and modifications that can be made without departing from principles underlying the invention, reference should be made to the appended claims for an understanding of the scope of the protection to be afforded the invention.

What is claimed is:

1. A method for improving lubricants employed in hydroforming processes comprising adding to said lubricants at least one overbased detergent and at least one friction modifier.

2. The method of claim 1 wherein the overbased detergent is selected from the group consisting of sulfonate, carboxylate, phenate, salicylate, and mixtures thereof.

3. The method of claim 2 wherein the overbased detergent is overbased sulfonate.

4. The method of claim 1 wherein the friction modifier is selected from the group consisting of fatty acid esters, reaction products of fatty acid esters and ethoxylated amines, overbased carboxylic acids, molybdenum dithiocarbamate derivatives, and mixtures thereof.

5. The method of claim 2 wherein the friction modifier is selected from the group consisting of fatty acid esters, reaction products of fatty acid esters and ethoxylated amines, overbased carboxylic acids, molybdenum dithiocarbamate derivatives, and mixtures thereof.

6. The method of claim 3 wherein the friction modifier is selected from the group consisting of fatty acid esters, reaction products of fatty acid esters and ethoxylated amines, overbased carboxylic acids, molybdenum dithiocarbamate derivatives, and mixtures thereof.

7. A lubricant for use in hydroforming processes wherein said lubricant comprises at least one overbased detergent and at least one friction modifier.

8. The lubricant of claim 7 wherein the overbased detergent is selected from the group consisting of sulfonate, carboxylate, phenate, salicylate, and mixtures thereof.

9. The lubricant of claim 8 wherein the overbased detergent is overbased sulfonate.

10. The lubricant of claim 7 wherein the friction modifier is selected from the group consisting of fatty acid esters, reaction products of fatty acid esters and ethoxylated amines, overbased carboxylic acids, molybdenum dithiocarbamate derivatives, and mixtures thereof.

11. The lubricant of claim 8 wherein the friction modifier is selected from the group consisting of fatty acid esters, reaction products of fatty acid esters and ethoxylated amines, overbased carboxylic acids, molybdenum dithiocarbamate derivatives, and mixtures thereof.

12. The lubricant of claim 9 wherein the friction modifier is selected from the group consisting of fatty acid esters, reaction products of fatty acid esters and ethoxylated amines, overbased carboxylic acids, molybdenum dithiocarbamate derivatives, and mixtures thereof.

13. In a method for hydroforming metal tubes or sheets, the improvement comprising employing in said method a lubricant comprising at least one overbased detergent and at least one friction modifier.

14. The method of claim 13 wherein the overbased detergent is selected from the group consisting of sulfonate, carboxylate, phenate, salicylate, and mixtures thereof.

15. The method of claim 14 wherein the overbased detergent is overbased sulfonate.

16. The method of claim 13 wherein the friction modifier is selected from the group consisting of fatty acid esters, reaction products of fatty acid esters and ethoxylated amines, overbased carboxylic acids, molybdenum dithiocarbamate derivatives, and mixtures thereof.

17. The method of claim 14 wherein the friction modifier is selected from the group consisting of fatty acid esters, reaction products of fatty acid esters and ethoxylated amines, overbased carboxylic acids, molybdenum dithiocarbamate derivatives, and mixtures thereof.

18. The method of claim 15 wherein the friction modifier is selected from the group consisting of fatty acid esters, reaction products of fatty acid esters and ethoxylated amines, overbased carboxylic acids, molybdenum dithiocarbamate derivatives, and mixtures thereof.
